(19) United States
(12) Patent Application Publication
(10) Pub. No.: US 2007/0066566 A1
(43) Pub. Date: Mar. 22, 2007

(54) 19,23,24,25,26,27-Hexanor-1alpha-hydroxyvitamin D3

(75) Inventors: Hector F. DeLuca, Deerfield, WI (US); Pawel Grzywacz, Madison, WI (US); Lori A. Plum, Arena, WI (US); Margaret Clagett-Dame, Deerfield, WI (US)

Correspondence Address: FOLEY & LARDNER LLP, 150 EAST GILMAN STREET, P.O. BOX 1497, MADISON, WI, 53701-1497, US

(73) Assignee: Wisconsin Alumni Research Foundation

(21) Appl. No.: 11/523,750

(22) Filed: Sep. 19, 2006

Related U.S. Application Data
(60) Provisional application No. 60/719,374, filed on Sep. 22, 2005.

(51) Int. Cl. A61K 31/695; A61K 31/59; C07C 401/00

(52) U.S. Cl. 514/63; 514/167; 552/653

(57) ABSTRACT

A compound or composition is provided that comprises a compound of formula 1A, 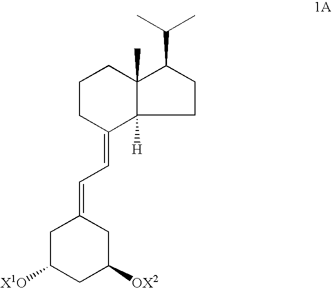
where, X¹ and X² are independently selected from H or hydroxy-protecting groups. Also provided are compounds that form the compound of formula 1A after they are administered to a subject.

### CROSS REFERENCES TO RELATED APPLICATIONS

This application claims priority to U.S. Provisional Application No. 60/719,374, filed Sep. 22, 2005, and incorporated by reference in its entirety and for all purposes as if fully set forth herein.

### FIELD OF THE INVENTION

This invention relates to 19-nor analogs of 1α,25-dihydroxyvitamin D₃ such as 19,23,24,25,26,27-hexanor-1α-hydroxyvitamin D₃ and analogs thereof, and to pharmaceutical formulations that include this compound or analogs thereof. The invention also relates to the use of the compounds, and mixtures thereof in the preparation of medicaments for use in treating various diseases such as skin conditions.

### BACKGROUND OF THE INVENTION

The natural hormone, 1α,25-dihydroxyvitamin D₃ (also referred to as 1α,25-dihydroxycholecalciferol and calcitriol) and its analog in the ergosterol series, i.e. 1α,25-dihydroxyvitamin D₂ are known to be highly potent regulators of calcium homeostasis in animals and humans, and their activity in cellular differentiation has also been established, Ostrem et al., Proc. Natl. Acad. Sci. USA, 84, 2610 (1987). Many structural analogs of these metabolites have been prepared and tested, including 1α-hydroxyvitamin D₃, 1α-hydroxyvitamin D₂, various side chain homologated vitamins, and fluorinated analogs. Some of these compounds exhibit an interesting separation of activities in cell differentiation and calcium regulation. This difference in activity may be useful in the treatment of a variety of diseases as renal osteodystrophy, vitamin D-resistant rickets, osteoporosis, psoriasis, and certain malignancies. The structure of 1α,25-dihydroxyvitamin D₃ and the numbering system used to denote the carbon atoms in this compound are shown below.

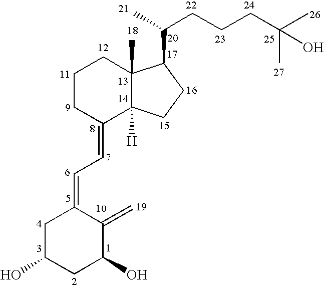

1α,25-Dihydroxyvitamin D₃=1α,25-Dihydroxycholecalciferol=Calcitriol

Another class of vitamin D analogs, i.e. the so called 19-nor-vitamin D compounds, is characterized by the replacement of the A-ring exocyclic methylene group (carbon 19), typical of the vitamin D system, by two hydrogen atoms. Biological testing of such 19-nor-analogs (e.g., 1α,25-dihydroxy-19-nor-vitamin D₃) revealed a selective activity profile with high potency in inducing cellular differentiation, and very low calcium mobilizing activity. Thus, these compounds are potentially useful as therapeutic agents for the treatment of malignancies, or the treatment of various skin disorders. Two different methods of synthesis of such 19-nor-vitamin D analogs have been described (Perlman et al., Tetrahedron Lett. 31, 1823 (1990); Perlman et al., Tetrahedron Lett. 32, 7663 (1991), and DeLuca et al., U.S. Pat. No. 5,086,191).

Various 2-substituted analogs of 1α,25-dihydroxy-19-nor-vitamin D₃ have also been synthesized, i.e. compounds substituted at the 2-position with hydroxy or alkoxy groups (DeLuca et al., U.S. Pat. No. 5,536,713), with 2-alkyl groups (DeLuca et al., U.S. Pat. No. 5,945,410), and with 2-alkylidene groups (DeLuca et al., U.S. Pat. No. 5,843,928), which exhibit interesting and selective activity profiles. All these studies indicate that binding sites in vitamin D receptors can accommodate different substituents at C-2 in the synthesized vitamin D analogs.

U.S. Pat. No. 4,666,634 discloses 2β-hydroxy and alkoxy (e.g., ED-71) analogs of 1α,25-dihydroxyvitamin D₃ as potential drugs for use in treating osteoporosis and for use as antitumor agents. See also Okano et al., Biochem. Biophys. Res. Commun. 163, 1444 (1989). Other 2-substituted (with hydroxyalkyl, e.g., ED-120, and fluoroalkyl groups) A-ring analogs of 1α,25-dihydroxyvitamin D₃ have been prepared and tested (Miyamoto et al., Chem. Pharm. Bull. 41, 1111 (1993); Nishii et al., Osteoporosis Int. Suppl. 1, 190 (1993); Posner et al., J. Org. Chem. 59, 7855 (1994), and J. Org. Chem. 60, 4617 (1995)).

In a continuing effort to explore the 19-nor class of pharmacologically important vitamin D compounds, their analogs which are characterized by the transposition of the ring A exocyclic methylene group from carbon 10 (C-10) to carbon 2 (C-2), i.e. 2-methylene-19-nor-vitamin D compounds have been recently synthesized and tested (Sicinski et al., J. Med. Chem., 41, 4662 (1998); Sicinski et al., Steroids 67, 247 (2002); DeLuca et al., U.S. Pat. Nos. 5,843,928, 5,936,133 and 6,382,071). Molecular mechanics studies, performed on these analogs, showed that a change of ring-A conformation can be expected resulting in the “flattening” of the cyclohexanediol ring. From molecular mechanics calculations and NMR studies of these compounds, the A-ring conformational equilibrium was established to be about 6:4 in favor of the conformer that has an equatorial 1α-OH. Introduction of the 2-methylene group into the 19-nor-vitamin D carbon skeleton changes the character of its (1α- and 3β-) A-ring hydroxyl groups; they are both now in the allylic positions, similar to the 1α-hydroxyl group (important for biological activity) in the natural hormone, 1α,25-(OH)₂D₃. 1α,25-Dihydroxy-2-methylene-19-norvitamin D analogs are characterized by significant biological potency which is enhanced in compounds with the “unnatural” (20S)-configuration.

In a continuing effort to explore the 19-nor class of pharmacologically important vitamin D compounds, analogs which are characterized by the presence of a methylene substituent at carbon 2 (C-2), a hydroxyl group at carbon 1 (C-1), and a shortened side chain attached to carbon 20 (C-20) have also been synthesized and tested. 1α-Hydroxy-2-methylene-19-nor-pregnacalciferol is described in U.S. Pat. No. 6,566,352 while 1α-hydroxy-2-methylene-19-nor-(20S)-homopregnacalciferol is described in U.S. Pat. No. 6,579,861 and 1α-hydroxy-2-methylene-19-nor-bishomopregnacalciferol is described in U.S. Pat. No. 6,627,622. All three of these compounds have relatively high binding activity to the vitamin D receptor and relatively high cell differentiation activity, but little if any calcemic activity as compared to 1α,25-dihydroxyvitamin D₃. Their biological activities make these compounds excellent candidates for a variety of pharmaceutical uses, as set forth in the '352, '861 and '622 patents.

A need exists for new biologically active analogs of vitamin D such as those described herein which are 19-nor analogs of 1α,25-dihydroxyvitamin D₃ with a shortened side chain attached to carbon 20.

### SUMMARY OF THE INVENTION

The invention provides 19-nor analogs of 1α,25-dihydroxyvitamin D₃ that have a shortened side chain such as 19,23,24,25,26,27-hexanor-1α-hydroxyvitamin D₃ and analogs thereof, pharmaceutical formulations or medicaments that include the compounds, and the use of these compounds or mixtures thereof in therapy and in the preparation of medicaments for use in treating various disease states.

Therefore, in one aspect, the invention provides compounds and compositions that include a compound having the formula 1A as shown below: 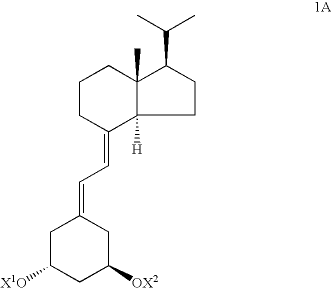
wherein,
X¹ and x² are independently selected from H or hydroxy-protecting groups. The invention also includes compounds that form a compound of formula 1A after it is administered to a subject.

In some embodiments, X¹ and X² are both hydroxy protecting groups such as silyl groups. In some such embodiments, X¹ and X² are both t-butyldimethylsilyl groups.

In some embodiments, X¹ and X² are both H such that the compound has the formula 1B as shown below:

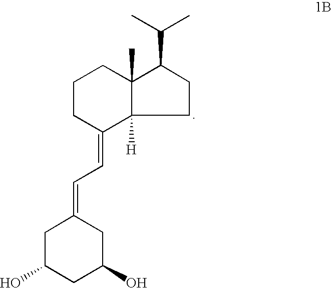

In some embodiments, the compounds of any of the embodiments may be present in a purified form. In other embodiments, the compounds in a composition may be present as a mixture.

The above compounds were/are tested and found to exhibit desired, and highly advantageous, patterns of biological activity with respect to ability to bind to the vitamin D receptor. The compounds may thus find use in therapy such as in treating cancer, skin conditions, and autoimmune disorders. Therefore, in some embodiments, these compounds or pharmaceutical formulations that include one or more compounds of the invention may be employed as therapeutic agents for the treatment of diseases or disorders such as cancer, autoimmune diseases, skin conditions, and secondary hyperparathyroidism. In some embodiments, the treatment may be transdermal, oral, or parenteral.

The compounds of the invention may also be especially suited for treatment and prophylaxis of human disorders which are characterized by an imbalance in the immune system, e.g., in autoimmune diseases, including multiple sclerosis, lupus, diabetes mellitus, host versus graft reaction, and rejection of transplants; and additionally, for the treatment of inflammatory diseases, such as rheumatoid arthritis, and asthma, as well as the improvement of bone fracture healing and improved bone grafts. Acne, alopecia, skin conditions such as dry skin (lack of dermal hydration), undue skin slackness (insufficient skin firmness), insufficient sebum secretion and wrinkles, and hypertension are other conditions which may be treated with the compounds of the invention.

The compounds described herein were also tested and found to moderate cell differentiation activity. Thus, these compounds may also be used as therapeutic agents for the treatment of psoriasis and/or as anti-cancer agents, especially against leukemia, colon cancer, breast cancer and prostate cancer. In some embodiments, the compounds and compositions of the invention are used to treat a biological condition selected from psoriasis; leukemia; colon cancer; breast cancer; prostate cancer; multiple sclerosis; lupus; diabetes mellitus; host versus graft reaction; rejection of organ transplants; an inflammatory disease selected from rheumatoid arthritis, asthma, eczema, or inflammatory bowel diseases; a skin condition selected from wrinkles, lack of adequate skin firmness, lack of adequate dermal hydration, or insufficient sebum secretion; or secondary hyperparathyroidism. The compounds of the invention find particular use in cosmetic applications and are thus particularly suited for treating any of the skin conditions described herein.

The compounds of the invention may be used to prepare pharmaceutical formulations or medicaments that include a compound or a mixture of the compounds of the invention in combination with a pharmaceutically acceptable carrier. In some embodiments, the compounds are used to prepare an aerosol which may include a glycol compound such as propylene glycol. Such pharmaceutical formulations and medicaments may be used to treat various biological disorders such as those described herein. Methods for treating such disorders typically include administering an effective amount of the compound, or an appropriate amount of a pharmaceutical formulation or a medicament that includes the compound, to a subject suffering from the biological disorder. In some embodiments, the subject is a mammal. In some such embodiments, the mammal is selected from a rodent, a primate, a bovine, an equine, a canine, a feline, an ursine, a porcine, a rabbit, or a guinea pig. In some such embodiments, the mammal is a rat or is a mouse. In some embodiments, the subject is a primate such as, e.g., a human. In some embodiments, the compounds are used to prepare an aerosol which may include a glycol compound such as propylene glycol.

In some embodiments of the methods of the invention, the compound or pharmaceutical composition is administered to the subject orally, rectally, parenterally, transdermally, or topically. In other embodiments, the compound or pharmaceutical formulations is administered in an aerosol which may be accomplished using an inhaler or a nebulizer.

The compounds may be present in a composition to treat the above-noted diseases and disorders in an amount from about 0.01 μg/gm to about 1 mg/gm of the composition, preferably from about 0.1 μg/gm to about 500 μg/gm of the composition, and may be administered by any route described herein in dosages of from about 0.01 μg/day to about 1 mg/day, preferably from about 0.1 μg/day to about 500 μg/day.

Further objects, features and advantages of the invention will be apparent from the following detailed description.

### BRIEF DESCRIPTION OF THE DRAWINGS

FIGS. 1-4 illustrate various biological activities of 19,23,24,25,26,27-hexanor-1α-hydroxyvitamin D₃ (referred to as “NP9” in the Figures) compared with those of the native hormone 1α,25-dihydroxyvitamin D₃ (referred to as “1,25(OH)₂D₃” in the Figures).

FIG. 1 is a graph comparing the relative activity of NP9 and 1,25(OH)₂D₃ to compete for binding with [³H]-1,25-(OH)₂-D₃ to the full-length recombinant rat vitamin D receptor.

FIG. 2 is a graph comparing the percent HL-60 cell differentiation as a function of the concentration of NP9 with that of 1,25(OH)₂D₃.

FIG. 3 is a graph comparing the in vitro transcription activity of NP9 with that of 1,25(OH)₂D₃.

FIGS. 4A and 4B are graphs comparing intestinal calcium transport (4A) and bone calcium mobilization (4B) of NP9 and 1,25(OH)₂D₃.

### DETAILED DESCRIPTION OF THE INVENTION

Generally, the invention provides 19-nor analogs of 1α,25-dihydroxyvitamin D₃ that have a shortened side chain such as 19,23,24,25,26,27-hexanor-1α-hydroxyvitamin D₃ and analogs thereof, pharmaceutical formulations or medicaments that include the compounds, and the use of these compounds or mixtures thereof in the preparation of medicaments for use in treating various disease states.

Therefore, in one aspect, the invention provides compounds and compositions that include a compound having the formula 1A as shown below: 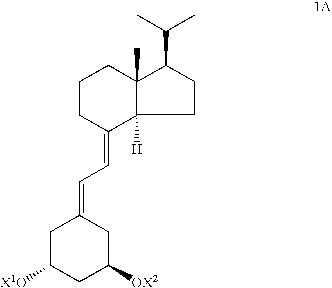
wherein,
X¹ and X² are independently selected from H or hydroxy-protecting groups. The invention also includes compounds that form a compound of formula 1A after it is administered to a subject.

In some embodiments, X¹ and X² are both hydroxy protecting groups such as silyl groups. In some such embodiments, X¹ and X² are both t-butyidimethylsilyl groups.

In some embodiments, X¹ and X² are both H such that the compound has the formula 1B as shown below:

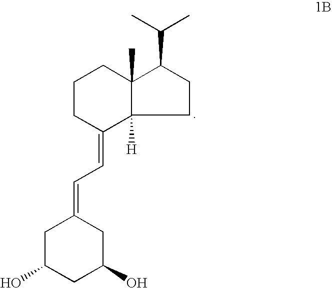

In some embodiments, the compounds of any of the embodiments may be present in a purified form. In other embodiments, the compounds in a composition may be present as a mixture.

As used herein, the term “hydroxy-protecting group” signifies any group commonly used for the temporary protection of the hydroxy (—OH) functional group, such as, but not limited to, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groups such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term “acyl” signifies an alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halo, nitro or alkyl substituted benzoyl group. Alkoxyalkyl protecting groups are groups such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. The term “aryl” specifies a phenyl-, or an alkyl-, nitro- or halo-substituted phenyl group. An extensive list of protecting groups for the hydroxy functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for all purposes as if fully set forth herein.

A “protected hydroxy” group is a hydroxy group derivatized or protected by any of the above groups commonly used for the temporary or permanent protection of hydroxy functional groups, e.g., the silyl, alkoxyalkyl, acyl or alkoxycarbonyl groups, as previously defined.

The compounds of the invention were prepared using the methods described herein. Reference should be made to the following description as well as to Schemes 1 and 2 for a detailed illustration of the preparation of the compounds of formula 1A and 1B and specifically 19,23,24,25,26,27-hexanor-1α-hydroxyvitamin D₃.

### EXAMPLES

The synthesis and characteristics of various 19-nor vitamin D analogs is described in numerous United States patents including U.S. Pat. No. 5,843,928, U.S. Pat. No. 5,616,759, U.S. Pat. No. 5,597,932, U.S. Pat. No. 5,281,731, U.S. Pat. No. 6,627,622, U.S. Pat. No. 6,579,861, U.S. Pat. No. 5,086,191, U.S. Pat. No. 5,585,369, and U.S. Pat. No. 6,537,981.

Melting points (uncorrected) were determined using a Thomas-Hoover capillary melting-point apparatus. Ultraviolet (UV) absorption spectra were recorded with a Perkin-Elmer Lambda 3B UV-VIS spectrophotometer in ethanol. ¹H nuclear magnetic resonance (NMR) spectra were recorded at 400 and 500 MHz using Bruker Instruments DMX-400 and DMX-500 Avance console spectrometers in CDCl₃. ¹³C nuclear magnetic resonance (NMR) spectra were recorded at 125 MHz with a Bruker Instruments DMX-500 Avance console spectrometer in CDCl₃. Chemical shifts (δ) are reported downfield from internal Me₄Si (δ 0.00). Electron impact (EI) mass spectra were obtained with a Micromass AutoSpec (Beverly, Mass.) instrument. High-performance liquid chromatography (HPLC) was performed on a Waters Associates liquid chromatograph equipped with a Model 6000A solvent delivery system, a Model U6K Universal injector, and a Model 486 tunable absorbance detector. THF was freshly distilled before use from sodium benzophenone ketyl under argon.

Schemes 1 and 2 outline the synthetic procedures described below, in detail.

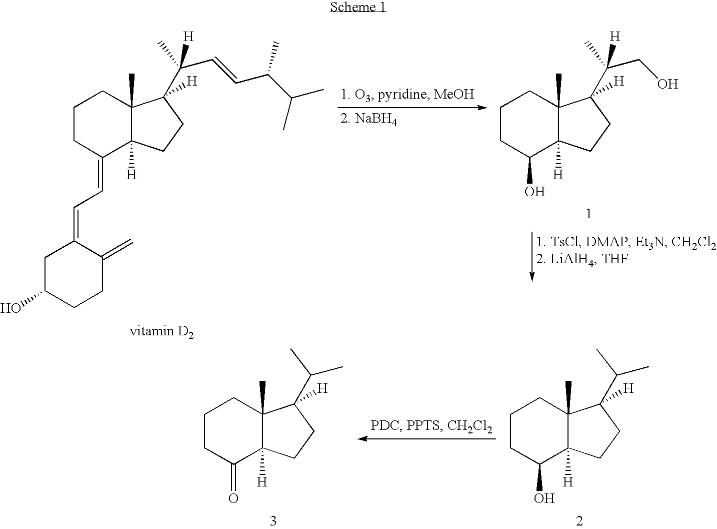

### Preparation of (20S)-De-A,B-8β-hydroxy-20-(hydroxymethyl)pregnane (1)

Ozone was passed through a solution of vitamin D₂ (3 g, 7.6 mmol) in methanol (250 mL) and pyridine (2.44 g, 2.5 mL, 31 mmol) for 50 minutes at −78° C. The reaction mixture was then flushed with oxygen for 15 minutes to remove the residual ozone and the solution was treated with NaBH₄ (0.75 g, 20 mmol). After 20 minutes, the second portion of NaBH₄ (0.75 g, 20 mmol) was added, and the mixture was allowed to warm to room temperature. The third portion of NaBH₄ (0.75 g, 20 mmol) was then added, and the reaction mixture was stirred for 18 hours. The reaction was quenched with water (40 mL), and the solution was concentrated under reduced pressure. The residue was extracted with ethyl acetate (3×80 mL), and the combined organic phase was washed with 1M aq. HCl, washed with saturated aq. NaHCO₃, dried (Na₂SO₄) and concentrated under reduced pressure. The residue was chromatographed on silica gel with hexane/ethyl acetate (75:25) to give the product 1 (1.21 g, 75% yield) as white crystals.

1: m.p. 106-108° C.; [α]_{D}+30.2° (c 1.46, CHCl₃); ¹H NMR (400 MHz, CDCl₃) δ 4.08 (1H, d, J=2.0 Hz, 8α-H), 3.63 (1H, dd, J=10.5, 3.1 Hz, 22-H), 3.38 (1H, dd, J=10.5, 6.8 Hz, 22-H), 1.99 (1H, br.d, J=13.2 Hz), 1.03 (3H, d, J=6.6 Hz, 21-H₃), 0.956 (3H, s, 18-H₃); ¹³C NMR (100 MHz) δ 69.16 (d, C-8), 67.74 (t, C-22), 52.90 (d), 52.33 (d), 41.83 (s, C-13), 40.19 (t), 38.20 (d), 33.53 (t), 26.62 (t), 22.54 (t), 17.36 (t), 16.59 (q, C-21), 13.54 (q, C-18); MS (EI) m/z 212 (2, M⁺), 194 (34, M⁺-H₂O), 179 (33, M⁺-H₂O—CH₃), 163 (18, M⁺-CH₂OH—H₂O), 135 (36), 125 (54), 111 (100), 95 (63), 81 (67); exact mass calculated for C₁₃H₂₂O (M⁺-H₂O) 194.1671, found 194.1665.

### Preparation of De-A,B-20-methyl-pregnan-8β-ol (2)

To a stirred solution of the diol 1 (1 g, 4.7 mmol), DMAP (50 mg, 0.4 mmol) and Et₃N (1.96 mL, 1.42 g, 14.1 mmol) in anhydrous methylene chloride (25 mL), was added p-toluenesulfonyl chloride (1.07 g, 5.6 mmol) at 0° C. The reaction mixture was stirred at 0° C. for 22 hours. Methylene chloride (60 mL) was added, and the mixture was washed with water, dried (Na₂SO₄), and concentrated under reduced pressure. The residue was chromatographed on silica gel with hexane/ethyl acetate (9:1, then 85:15) to afford a tosylate (1.72 g, 100% yield) as a colorless oil.

Lithium aluminum hydride (360 mg, 9.5 mmol) was added to a solution of the tosylate (1.72 g, 4.7 mmol) in anhydrous THF (25 mL) at 0° C. The cooling bath was removed, and the reaction mixture was stirred at room temperature for 5 hours. The excess hydride was quenched by careful, successive addition of methanol at 0° C. A saturated aq. solution of tartaric acid was added, and the mixture was extracted with ethyl acetate. The combined organic phase was washed with water, dried (Na₂SO₄) and concentrated under reduced pressure. The residue was chromatographed on silica gel with hexane/ethyl acetate (9:1, 8:2) to give the alcohol 2 (811 mg, 88%).

2: [α]_{D}+25.3° (c 1.48, CHCl₃); ¹H NMR (400 MHz, CDCl₃+TMS) δ 4.07 (1H, d, J=2.2 Hz, 8α-H), 1.98 (1H, br.d, J=13.4 Hz), 0.928 (3H, s, 18-H₃), 0.92 and 0.84 (each 3H, each d, each J=6.6 Hz, 21-H₃ and 22-H₃); ¹³C NMR (100 MHz) δ 69.39 (d, C-8), 58.72 (d), 52.57 (d), 41.80 (s, C-13), 40.24 (t), 33.52 (t), 30.52 (d), 27.34 (t), 23.02 (q, C-21 or C-22), 22.51 (t), 22.34 (q, C-21 or C-22), 17.40 (t), 13.61 (q, C-18); MS (EI) m/z 196 (24, M⁺), 181 (30, M⁺-CH₃), 163 (11, M⁺-H₂O—CH₃), 135 (20, M⁺-H₂O—C₃H₇), 125 (26), 111 (100), 97 (33), 82 (57); exact mass calculated for C₁₃H₂₄O (M⁺) 196.1827, found 196.1821.

### Preparation of De-A,B-20-methyl-pregnan-8-one (3)

Pyridinium dichromate (720 mg, 1.9 mmol) was added to a solution of alcohol 2 (94 mg, 0.48 mmol) and pyridinium p-toluenesulfonate (5 mg, 20 μmol) in anhydrous methylene chloride (6 mL). The resulting suspension was stirred at room temperature for 4 hours. The reaction mixture was filtered through a Waters silica Sep-Pak cartridge (2 g) that was further washed with CH₂Cl₂. After removal of solvents, ketone 3 (86 mg, 92% yield) was obtained as a colorless oil.

3: [α]_{D}−28.8° (c 1.09, CHCl₃); ¹H NMR (400 MHz, CDCl₃) δ2.43 (1H, dd, J=11.5, 7.6 Hz), 0.95 and 0.86 (each 3H, each d, each J=6.5 Hz, 21-H₃ and 22-H₃), 0.617 (3H, s, 18-H₃); ¹³C NMR (100 MHz) δ 212.11 (s, C-8), 61.93 (d) 58.61 (d), 49.90 (s, C-13), 40.90 (t), 38.82 (t), 30.70 (d), 27.60 (t), 24.03 (t), 22.95 and 22.45 (each q, C-21 and C-22), 19.02 (t), 12.51 (q, C-18); MS (EI) m/z 194 (63, M⁺), 179 (74, M⁺-CH₃), 151 (100, M⁺-C₃H₇), 133 (22), 125 (58), 111 (48), 96 (85), 81 (80); exact mass calculated for C₁₃H₂₂O (M⁺) 194.1671, found 194.1664.

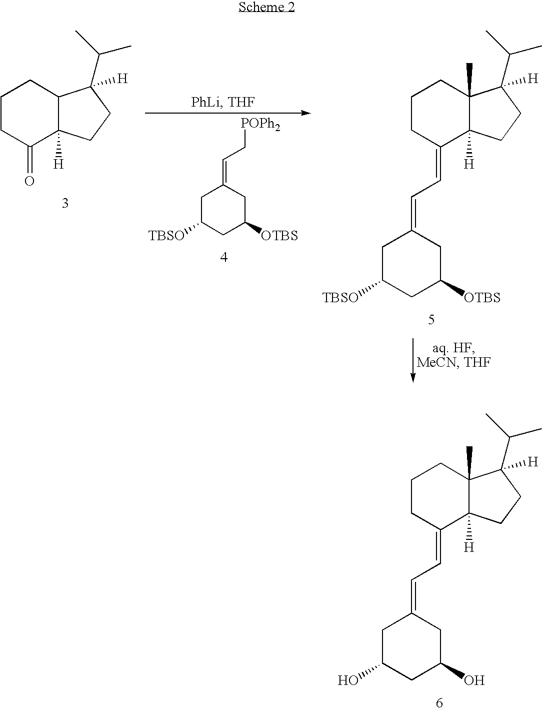

### Preparation of 19,23,24,25,26,27-Hexanor-1α-hydroxyvitamin D₃ (6))

To a solution of phosphine oxide 4 (250 mg, 0.44 mmol) in anhydrous THF (1 mL) at −20° C. was slowly added PhLi (1.3 M in cyclohexane-ether, 400 μL, 0.52 mmol) under argon with stirring (See Perlman et al., Tetrahedron Lett. 32, 7663 (1991); and U.S. Pat. No. 5,616,759 issued to DeLuca et al. both of which are hereby incorporated by reference in their entireties as if fully set forth herein). The solution turned deep orange. After 30 minutes, the mixture was cooled to −78° C. and a precooled (−78° C.) solution of ketone 3 (86 mg, 0.44 mmol) in anhydrous THF (600+200 μL) was slowly added. The mixture was stirred under argon at −78° C. for 6 hours and at 0° C. for 18 hours. Ethyl acetate was added, and the organic phase was washed with brine, dried (Na₂SO₄) and evaporated. The residue was dissolved in hexane and applied on a Waters silica Sep-Pak cartridge (5 g). The cartridge was washed with hexane and hexane/ethyl acetate (99.5:0.5) to give 19-norvitamin derivative 5 (180 mg, 0.33 mmol, 92% yield). The Sep-Pak was then washed with hexane/ethyl acetate (96:4) to recover the unchanged C,D-ring ketone 3 (15 mg, 0.08 mmol), and with ethyl acetate to recover diphenylphosphine oxide 4 (52 mg).

5: UV (in hexane) λₘₐₓ 261.7, 252.0, 243.2 nm; ¹H NMR (500 MHz, CDCl₃) δ 6.18 and 5.83 (1H and 1H, each d, J=11.2 Hz, 6- and 7-H), 4.10 (2H, m, 1β- and 3α-H), 2.82 (1H, br d, J=12.4 Hz, 9-H), 2.38 (2H, m, 4-H and 10-H), 2.27 (1H, br d, J=13.5 Hz, 10-H), 2.11 (1H, dd, J=12.8, 8.1 Hz, 4-H), 1.99 (2H, m), 0.96 (3H, d, J=6.5 Hz, 21- or 22-H₃), 0.887 (9H, s, Si-t-Bu), 0.874 (9H, s, Si-t-Bu), 0.87 (3H, d, J=6.5 Hz, 21- or 22-H₃), 0.547 (3H, s, 18-H₃), 0.069, 0.063, 0.059 and 0.058 (each 3H, each s, 4×Si—CH₃); ¹³C NMR (100 MHz) δ 140.73 (s, C-8), 133.52 (s, C-5), 121.77 (d, C-6), 116.12 (d, C-7), 68.10 and 67.96 (each d, C-1 and C-3), 58.61 (d), 56.25 (d), 46.00 (t), 45.61 (s, C-13), 43.73 (t), 40.51 (t), 36.80 (t), 31.34 (d), 28.67 (t), 27.83 (t), 25.85 (q, 2×SiCMe₃), 23.41 (t), 23.14 and 22.66 (each q, C-21 and C-22), 22.23 (t), 18.09 (s, 2×SiCMe₃), 12.14 (q, C-18), −4.66 (q, SiCMe), −4.76 (q, SiCMe), −4.83 (q, SiCMe), −4.90 (q, SiCMe); MS (EI) m/z 546 (46, M⁺), 489 (28, M⁺-t-Bu), 414 (100, M⁺-t-BuMe₂SiOH), 357 (48, M⁺-t-BuMe₂SiOH-t-Bu), 301 (21), 135 (20), 73 (91); exact mass calculated for C₃₃H₆₂O₂Si₂ (M⁺) 546.4288, found 546.4295.

Protected vitamin 5 (180 mg, 0.33 mmol) was dissolved in THF (3 mL) and acetonitrile (2 mL). A solution of aqueous 48% HF in acetonitrile (1:9 ratio, 5 mL) was added at 0° C. and the resulting mixture was stirred at room temperature for 3 hours. Saturated aqueous NaHCO₃ solution was added, and the reaction mixture was extracted with ethyl acetate. The combined organic phase was washed with brine, dried (Na₂SO₄), and concentrated under reduced pressure. The residue was diluted with 2 mL of hexane/ethyl acetate (95:5) and applied on a Waters silica Sep-Pak cartridge (5 g). An elution with hexane/ethyl acetate (1:1) gave the crude product 6 (102 mg). The vitamin 6 was further purified by straight phase HPLC [20×250 mm Zorbax Pro-10 SIL column, 14 mL/min, hexane/2-propanol (85:15) solvent system, Rₜ=10.00 min.] and later by reverse phase HPLC [30×250 mm Luna 5u C18(2) column, 15 mL/min, methanol/water (97:3) solvent system, Rₜ=14.82 min.] to give a colorless oil (70.72 mg, 67% yield).

6: UV (in EtOH) λₘₐₓ 260.8, 251.2, 242.6 nm; ¹H NMR (500 MHz, CDCl₃) δ 6.30 and 5.85 (1H and 1H, each d, J=11.2 Hz, 6- and 7-H), 4.11 (1H, m, 3α-H), 4.04 (1H, m, 1β-H), 2.79 (1H, dd, J=12.2, 4.0 Hz, 9-H), 2.73 (1H, dd, J=13.2, 3.7 Hz, 10-H), 2.48 (1H, dd, J=13.3, 3.3 Hz, 4-H), 2.21 (2H, m, 4-H and 10-H), 0.94 (3H, d, J=6.6 Hz, 21- or 22-H₃), 0.86 (3H, d, J=6.6 Hz, 21- or 22-H₃), 0.534 (3H, s, 18-H₃); ¹³C NMR (125 MHz) δ 142.55 (s, C-8), 131.56 (s, C-5), 123.28 (d, C-6), 115.27 (d, C-7), 67.03 and 66.71 (each d, C-1 and C-3), 58.46 (d), 56.15 (d), 45.61 (s, C-13), 44.27 (t), 41.88 (t), 40.22 (t), 36.83 (t), 31.20 (d), 28.77 (t), 27.66 (t), 23.37 (t), 23.01 and 22.52 (each q, C-21 and C-22), 22.17 (t), 12.05 (q, C-18). MS (EI) m/z 318 (100, M⁺), 303 (6, M⁺-Me), 275 (41, M⁺-C₃H₇), 257 (15, M⁺-C₃H₇—H₂O), 239 (19, M⁺-C₃H₇-2H₂O), 221 (23), 203 (18), 189 (39), 177 (58), 147 (33), 135 (46), 123 (39), 105 (36), 95 (55); exact mass calculated for C₂₁H₃₄O₂ (M⁺) 318.2559, found 318.2547.

### Biological Activity

### Vitamin D Receptor Binding

Test Material

Protein Source

Full-length recombinant rat receptor was expressed in E. coli BL21(DE3) Codon Plus RIL cells and purified to homogeneity using two different column chromatography systems. The first system was a nickel affinity resin that utilizes the C-terminal histidine tag on this protein. The protein that eluted from this resin was further purified using ion exchange chromatography (S-Sepharose Fast Flow). Aliquots of the purified protein were quick frozen in liquid nitrogen and stored at −80° C. until use. For use in binding assays, the protein was diluted in TEDK₅₀ (50 mM Tris, 1.5 mM EDTA, pH 7.4, 5 mM DTT, 150 mM KCl) with 0.1% Chaps detergent. The receptor protein and ligand concentration was optimized such that no more than 20% of the added radiolabeled ligand is bound to the receptor.

Study Drugs

Unlabeled ligands were dissolved in ethanol and the concentrations were determined using UV spectrophotometry (1,25(OH)₂D₃: molar extinction coefficient=18,200 and λₘₐₓ=265 nm). Radiolabeled ligand (³H-1,25(OH)₂D₃, ˜159 Ci/mmol) was added in ethanol at a final concentration of 1 nM.

Assay Conditions

Radiolabeled and unlabeled ligands were added to 100 mcl of the diluted protein at a final ethanol concentration of ≧10%, mixed and incubated overnight on ice to reach binding equilibrium. The following day, 100 mcl of hydroxylapatite slurry (50%) was added to each tube and was mixed at 10-minute intervals for 30 minutes. The hydroxylapaptite was collected by centrifugation and was then washed three times with Tris-EDTA buffer (50 mM Tris, 1.5 mM EDTA, pH 7.4) containing 0.5% Titron X-100. After the final wash, the pellets were transferred to scintillation vials containing 4 mL of Biosafe II scintillation cocktail, mixed and placed in a scintillation counter. Total binding was determined from the tubes containing only radiolabeled ligand.

### HL-60 Differentiation

Test Material

Study Drugs

The study drugs were dissolved in ethanol and the concentrations determined using UV spectrophotometry. Serial dilutions were prepared so that a range of drug concentrations was tested without changing the final concentration of ethanol (≦0.2%) present in the cell cultures.

Cells

Human promyelocytic leukemia (HL60) cells were grown in RPMI-1640 medium containing 10% fetal bovine serum. The cells were incubated at 37° C. in the presence of 5% CO₂.

Assay Conditions

HL60 cells were plated at 1.2×10⁵ cells/mL. Eighteen hours after plating, cells in duplicate were treated with drug. Four days later, the cells were harvested and a nitro blue tetrazolium reduction assay was performed (Collins et al., 1979; J. Exp. Med. 149:969-974). The percentage of differentiated cells was determined by counting a total of 200 cells and recording the number that contain intracellular black-blue formazan deposits. Verification of differentiation to monocytic cells was determined by measuring phagocytic activity.

### In Vitro Transcription Assay

Transcription activity was measured in ROS 17/2.8 (bone) cells that were stably transfected with a 24-hydroxylase (24Ohase) gene promoter upstream of a luciferase reporter gene (Arbour et al., 1998). Cells were given a range of doses. Sixteen hours after dosing the cells were harvested and luciferase activities were measured using a luminometer. RLU=relative luciferase units.

Antagonism was tested by adding a combination of 1,25(OH)₂D₃ and the compound in the same well keeping the final ethanol concentration the same.

### Intestinal Calcium Transport and Bone Calcium Mobilization

Male, weanling Sprague-Dawley rats were placed on Diet 11 (Suda et al. J. Nutr. 100:1049, 1970) (0.47% Ca) diet+vitamins AEK for one week followed by Diet 11 (0.02% Ca)+AEK for 3 weeks. The rats were then switched to a diet containing 0.47% Ca for one week followed by two weeks on a diet containing 0.02% Ca. Dose administration began during the last week on 0.02% calcium diet. Four consecutive ip doses were given approximately 24 hours apart. Twenty-four hours after the last dose, blood was collected from the severed neck and the concentration of serum calcium was determined as a measure of bone calcium mobilization. The first 10 cm of the intestine was also collected for intestinal calcium transport analysis using the everted gut sac method. Antagonism was tested by administering a combination of 1,25(OH)₂D₃ and the compound to the animal simultaneously.

The compounds of the invention were prepared and studied using the methods described above. The compounds were found to exhibit desired, and highly advantageous, patterns of biological activity with respect to intestinal calcium transport activity, ability to mobilize calcium from bone, and ability to bind to the vitamin D receptor. The compounds are also found to moderate cell differentiation activity.

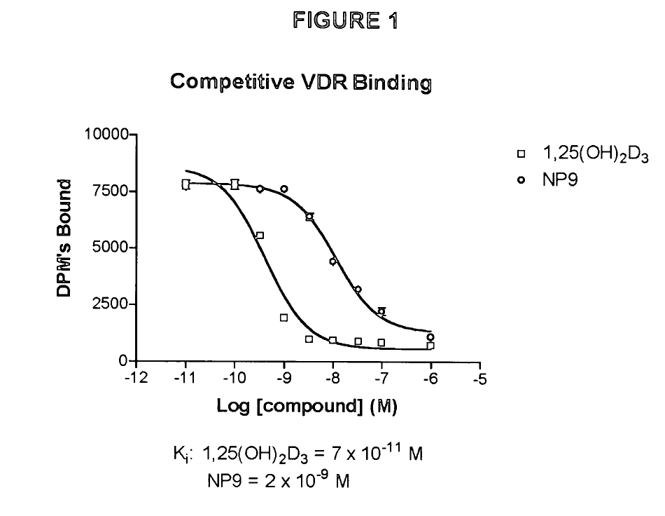

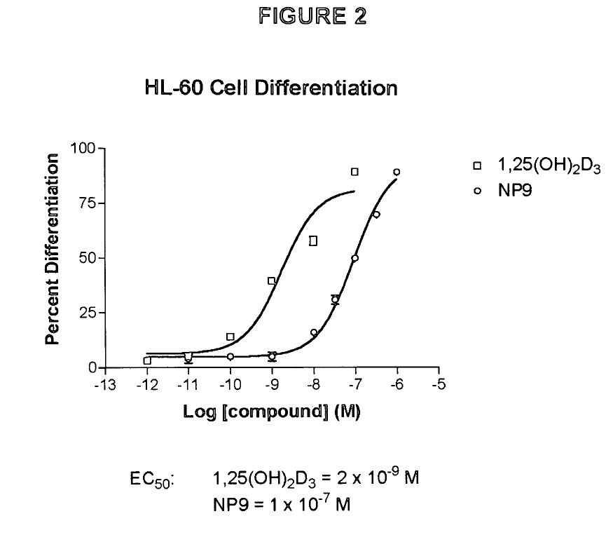

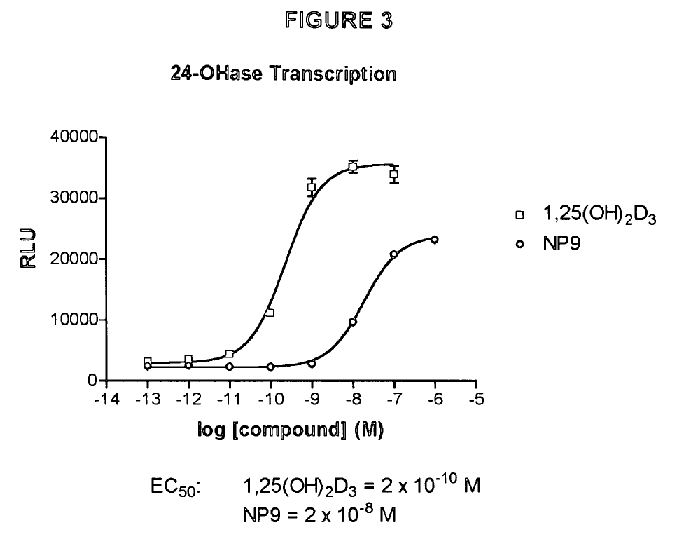

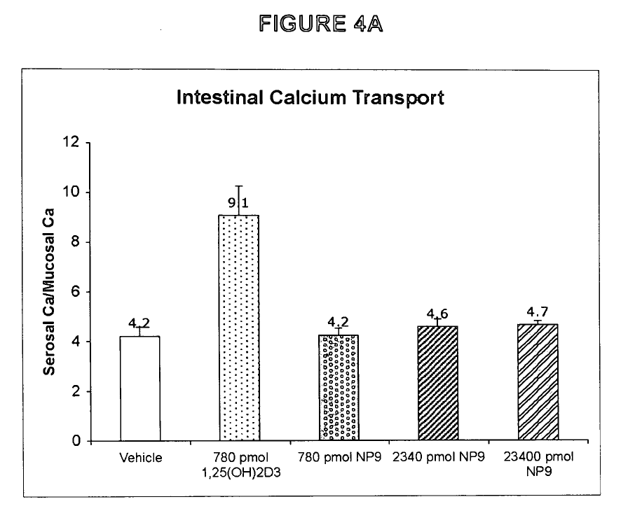

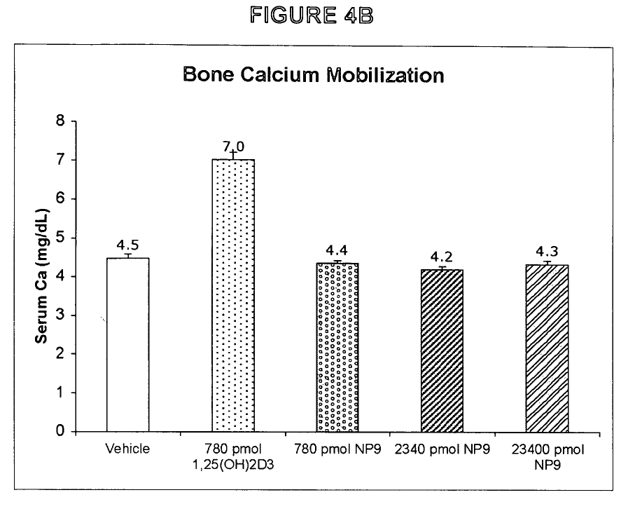

The compound of formula 1B does not bind to the vitamin D receptor as strongly as the native hormone 1,25-(OH)₂D₃ as shown in FIG. 1. The compound of formula 1B shows less but still significant activity compared to 1,25-(OH)₂D₃ in inducing differentiation of HL-60 cells (FIG. 2). The compound of formula 1B does not show as much activity in causing transcription as 1,25-(OH)₂D₃ as shown in FIG. 3. Finally, the compound of formula 1B has no measurable bone calcium mobilizing activity or intestinal calcium transport activity (FIGS. 4A and 4B).

For treatment purposes, the compounds of the invention may be formulated for pharmaceutical applications as a solution in innocuous solvents, or as an emulsion, suspension or dispersion in suitable solvents or carriers, or as pills, tablets or capsules, together with solid carriers, according to conventional methods known in the art. Any such formulations may also contain other pharmaceutically acceptable and non-toxic excipients such as stabilizers, anti-oxidants, binders, coloring agents or emulsifying or taste-modifying agents. Pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is hereby incorporated by reference in its entirety and for all purposes as if fully set forth herein.

The compounds may be administered orally, topically, parenterally, rectally, or transdermally. The compounds are advantageously administered by injection or by intravenous infusion or suitable sterile solutions, or in the form of liquid or solid doses via the alimentary canal, or in the form of creams, ointments, patches, or similar vehicles suitable for transdermal applications. In some embodiments, doses of from 0.001 μg to about 1 mg per day of the compound are appropriate for treatment purposes. In some such embodiments an appropriate and effective dose may range from 0.01 μg to 1 mg per day of the compound. In other such embodiments an appropriate and effective dose may range from 0.1 μg to 500 μg per day of the compound. Such doses will be adjusted according to the type of disease or condition to be treated, the severity of the disease or condition, and the response of the subject as is well understood in the art. The compound may be suitably administered alone, or together with another active vitamin D compound.

Compositions for use in the invention include an effective amount of a compound of any of the embodiments as the active ingredient or ingredients, and a suitable carrier. An effective amount of the compound or compounds for use in accordance with some embodiments of the invention will generally be a dosage amount such as those described herein, and may be administered topically, transdermally, orally, nasally, rectally, or parenterally.

Dosages as described above are suitable, it being understood that the amounts given are to be adjusted in accordance with the severity of the disease, and the condition and response of the subject as is well understood in the art. As noted, the compounds of the invention may be present as a mixture of two or more compounds. In some mixtures, the mixture may include a first compound of the invention and a second compound of the invention. In some embodiments, the mixture includes the first compound and the second compound, and the ratio of the first compound to the second compound ranges from 50:50 to 99.9:0.1. In some such embodiments, the ratio of the first compound to the second compound ranges from 70:30 to 99.9:0.1, from 80:20 to 99.9:0.1, from 90:10 to 99.9:0.1, or from 95:5 to 99.9:0.1.

The compound or compounds may be formulated as creams, lotions, ointments, aerosols, suppositories, topical patches, pills, capsules or tablets, or in liquid form as solutions, emulsions, dispersions, or suspensions in pharmaceutically innocuous and acceptable solvent or oils, and such preparations may contain, in addition, other pharmaceutically innocuous or beneficial components, such as stabilizers, antioxidants, emulsifiers, coloring agents, binders or taste-modifying agents.

The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof.

Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

Formulations for rectal administration may be in the form of a suppository incorporating the active ingredient and carrier such as cocoa butter, or in the form of an enema.

Formulations suitable for parenteral administration conveniently comprise a sterile oily or aqueous preparation of the active ingredient which is preferably isotonic with the blood of the recipient.

Formulations suitable for topical administration include liquid or semi-liquid preparations such as liniments, lotions, applicants, oil-in-water or water-in-oil emulsions such as creams, ointments or pastes; or solutions or suspensions such as drops; or as sprays.

For nasal administration, inhalation of powder, self-propelling or spray formulations, dispensed with a spray can, a nebulizer or an atomizer can be used. The formulations, when dispensed, preferably have a particle size in the range of 10 to 100 microns.

The formulations may conveniently be presented in dosage unit form and may be prepared by any of the methods well known in the art of pharmacy. By the term “dosage unit” is meant a unitary, i.e., a single dose which is capable of being administered to a patient as a physically and chemically stable unit dose comprising either the active ingredient as such or a mixture of it with solid or liquid pharmaceutical diluents or carriers.

All references cited herein are specifically incorporated by reference in their entireties and for all purposes as if fully set forth herein.

It is understood that the invention is not limited to the embodiments set forth herein for illustration, but embraces all such forms thereof as come within the scope of the following claims.

What is claimed is:

1. A compound or composition comprising a compound of formula 1A, 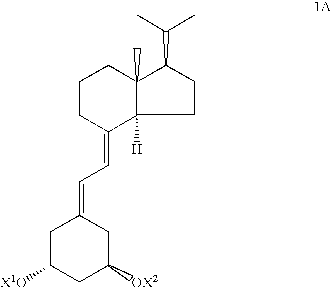
wherein,
X¹ and X² are independently selected from H or hydroxy-protecting groups, or a compound that forms the compound of formula IA in a subject after administration.

2. The compound or composition of claim 1, wherein X¹ and X² are both hydroxy protecting groups.

3. The compound or composition of claim 2, wherein X¹ and X² are both t-butyldimethylsilyl groups.

4. The compound or composition of claim 1, wherein X¹ and X² are both H such that the compound has the formula 1B

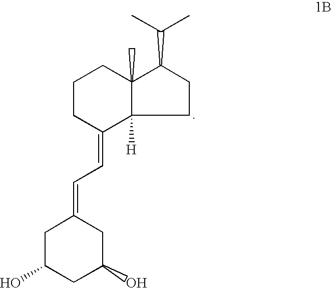

5. A pharmaceutical formulation, comprising: the compound of claim 1 and a pharmaceutically acceptable carrier.

6. The pharmaceutical formulation of claim 5, wherein the amount of the compound in the pharmaceutical formulation ranges from about 0.01 μg to about 1 mg per gram of the pharmaceutical formulation.

7. The pharmaceutical formulation of claim 6, wherein the amount of the compound in the pharmaceutical formulation ranges from about 0.1 μg to about 500 μg per gram of the pharmaceutical formulation.

8. The pharmaceutical formulation of claim 5, wherein X¹ and X² are both H such that the compound has the formula 1B

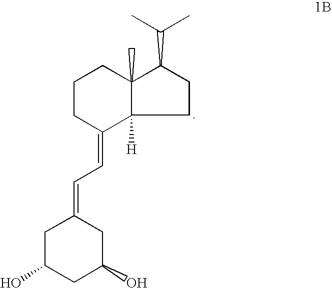

9. A method of treating a subject suffering from a biological disorder, comprising administering an effective amount of the compound or composition of claim 1 or a pharmaceutical formulation comprising the compound of claim 1 and a pharmaceutically acceptable carrier to the subject.

10. The method of claim 9, wherein the compound, the composition, or the pharmaceutical formulation is administered orally, parenterally, rectally, transdermally, or topically to the subject.

11. The method of claim 9, wherein the compound, the composition, or the pharmaceutical formulation is administered by delivering the compound, the composition, or the pharmaceutical formulation in an aerosol.

12. The method of claim 9, wherein the biological condition is selected from psoriasis; leukemia; colon cancer; breast cancer; prostate cancer; multiple sclerosis; lupus; diabetes mellitus; host versus graft reaction; rejection of organ transplants; an inflammatory disease selected from rheumatoid arthritis, asthma, eczema, or inflammatory bowel diseases; a skin condition selected from wrinkles, lack of adequate skin firmness, lack of adequate dermal hydration, or insufficient sebum secretion; or secondary hyperparathyroidism.

13. The method of claim 9, wherein the biological condition is a skin condition.

14. The method of claim 12, wherein the skin condition is selected from wrinkles, lack of adequate skin firmness, lack of adequate dermal hydration, eczema, psoriasis, or insufficient sebum secretion.

15. The method of claim 9, wherein X¹ and X² are both H such that the compound has the formula 1B [chemical structure]
